(19) United States
(12) Patent Application Publication
(10) Pub. No.: US 2005/0251075 A1
(43) Pub. Date: Nov. 10, 2005

(54) Devices for relieving pelvic discomfort

(76) Inventor: R. Scott Smith, Naples, FL (US)

Correspondence Address: HELLER EHRMAN WHITE & MCAULIFFE LLP, 1717 RHODE ISLAND AVE, NW, WASHINGTON, DC, 20036-3001, US

(21) Appl. No.: 11/178,416

(22) Filed: Jul. 12, 2005

Related U.S. Application Data
(63) Continuation of application No. 10/756,758, filed on Jan. 14, 2004, now abandoned.
Application No. 10/756,758 is a continuation of application No. 10/197,776, filed on Jul. 19, 2002, now abandoned.
(60) Provisional application No. 60/306,428, filed on Jul. 20, 2001.

(51) Int. Cl. A61F 5/00

(52) U.S. Cl. 602/19

(57) ABSTRACT

Devices are described that reduce pain associated with menstruation and other conditions and that operate by pressing upon the sacral/parasacral regions of a woman's lower back. In preferred embodiments a tensioning apparatus applies pressure onto a generally flat part that distributes force into the sacral and parasacral regions but avoids the lumbar region. The devices may assume a variety of forms such as belts, straps and foundation garments that can exert high pressures and may exceed 100 torr in some cases.

This application receives priority from U.S. Patent Application Ser. No. 60/306,428 entitled “Devices for Relieving Menstrual Discomfort” filed Jul. 20, 2001, and U.S. patent application Ser. No. 10/197,776 entitled “Devices for Relieving Pelvic Discomfort” filed on Jul. 19, 2002, the contents of which are incorporated by reference in their entirety.

### BACKGROUND OF THE INVENTION

1. Field of the Invention

The present invention relates generally to devices that are worn and that exert pressure on the body. More specifically, this invention relates to a pressure distributor that pushes specifically onto the sacral and/or parasacral regions of a woman's lower back to alter the physiological status of the woman.

2. Description of the Related Art

During and shortly prior to menstruation, many women suffer from pain and cramping in the pelvic area. These symptoms occur as a result of congestion in the pelvic area, which distorts the normal anatomy. Because the anatomy is distorted, normal neurovascular function is impaired, which in turn contributes to the discomfort. Related conditions such as pelvic inflammatory disease and endometriosis also may produce or accentuate pain and occur separately or sometimes in combination with menstruation.

Women have been using various girdle and the like type of garments for many years. However none have been particularly effective for relieving pain and cramping in the pelvic area such as that experienced prior to and during menstruation. In the art of applying fixed pressure to the body, a number of devices, primarily belts, have been proposed which operate on the back by applying pressure there. For example, U.S. Pat. No. 4,622,957 describes a “Therapeutic Corset” waist belt that applies pressure to a large portion of the back, including the sacrum, lumbar and thoracic regions, as shown in FIG. 5 of that patent. Unfortunately, devices such as those described in that patent do not alleviate pelvic pain such as menstrual pain sufficiently and can worsen pain by its generalized effect on other regions of the back.

This art has been advanced by technology described in U.S. Pat. No. 6,149,497, which illustrates use of a bladder in a foundation garment to apply pressure for alleviating menstrual pain. A related disclosure is found in PCT publication number WO 00/41656 entitled “Foundation Garment for the Relief of Menstrual Discomfort.” Continued advances in this field would bring useful relief to a common problem facing millions of women around the world.

### SUMMARY OF THE INVENTION

According to the present invention, the above and other objects and advantages are achieved by devices and methods for alleviating menstrual discomfort, endometriosis, pelvic inflammatory disease, and other conditions that produce pelvic pain. One embodiment of the invention is a device for alleviating pain such as menstrual pain in a woman, comprising a pressure distributor and a tensioning apparatus which can push the pressure distributor into the parasacral region and/or sacral region with at least 10 torr pressure while substantially not distributing force to the lumbar region of the woman's back. Another embodiment is a foundation garment that preferentially applies pressure to the sacral area and/or parasacral area, while substantially avoiding the lumbar area, comprising a conforming pressure distributor positioned over the sacral and/or parasacral region wherein the pressure distributor presses upon the parasacral area when worn by a woman in need of treatment for menstrual cramps.

Yet another embodiment of the invention is an improvement to a panty girdle, comprising adding a pressure distributor positioned over the sacral and or parasacral region that substantially avoids the lumbar region; and a means for adjusting pressure to the stiff portion while the panty girdle is worn. Another embodiment of the invention is a means for alleviating pelvic pain such as menstrual discomfort, endometriosis, pelvic inflammatory disease, and other conditions that produce pelvic pain in a woman, comprising continuously pressing the sacral and/or parasacral region of the woman's back while substantially avoiding the lumbar region, through a tensioning apparatus that is designed or modified for this purpose. Yet another embodiment of the invention is a device for alleviating pelvic pain in a woman, comprising a conforming and generally flat pressure distributor and a tensioning apparatus, wherein the tensioning apparatus holds and continuously pushes the conforming and generally flat portion into the sacral and parasacral regions.

These and other objects and advantages of the present invention are set forth in the description that follows, and in part will be readily apparent to those skilled in the art from the description and drawings, or may be learned by practice of the invention. These objects and advantages of the invention may be realized and obtained by means of the instrumentalities and combinations particularly pointed out in the appended claims.

### BRIEF DESCRIPTION OF THE DRAWINGS

The present invention will become more clearly appreciated in view of the following description of several preferred embodiments that relate to the appended drawings as listed here.

FIG. 1 is a front elevation view showing the anterior region of the area that is treated by embodiments of the invention.

FIG. 2 is a rear elevation view showing an area to which pressure can be applied by a pressure distributor according to an embodiment of the invention.

FIG. 3 is a side elevation view showing the direction in which the pressure distributor may apply pressure to the sacral and/or parasacral areas.

FIG. 4 is a top view of a compressible and stiff pressure distributor according to an embodiment of the invention.

FIG. 5 is a side view of the pressure distributor shown in FIG. 4.

FIG. 6 is a perspective view of the pressure distributor of FIG. 4.

FIG. 7 is an end view that shows a cross section of the pressure distributor of FIG. 4.

### DETAILED DESCRIPTION OF THE PREFERRED EMBODIMENT

Operation of the Invention

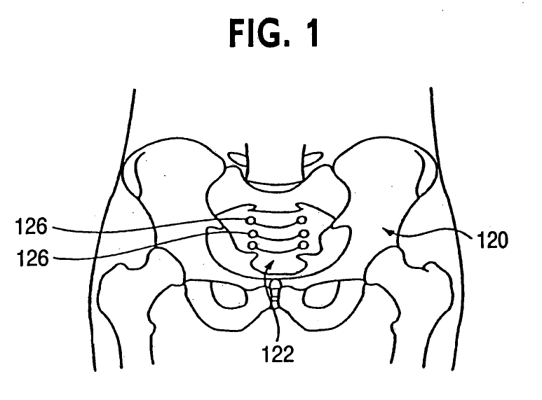

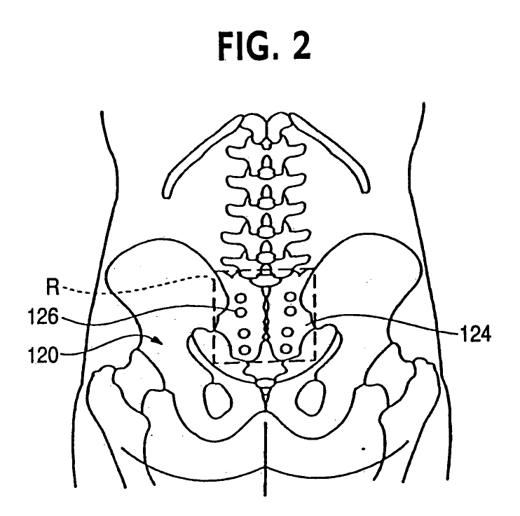

During menstruation and during some other diseases that may lead to pelvic pain, the pelvic organs and tissues become engorged with blood. This causes congestion due to edema and swelling of the entire pelvic areas 120 (see FIGS. 1 and 2). The congestion creates pressure posterior to the anterior sacrum 122 (FIG. 1) and sacral area 124 (seen from the back in FIG. 2). Nerves and veins in the sacral area 124 pass through small holes or sacral foramina 126 in this area, (FIGS. 1 and 2) and the pressure from the congestion distorts the normal anatomy, impeding neurologic function and venous drainage. The change in normal venous drainage exasperates the congestion, putting even more pressure on the nerve roots and causing additional discomfort.

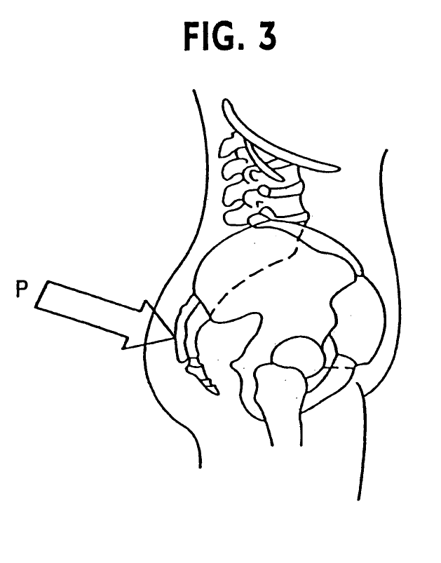

In reviewing the biological effects described above, devices and methods have been discovered that continuously and specifically press upon the posterior aspect of the sacrum and/or parasacral areas for relief of congestion and associated discomfort of the painful condition. The externally applied pressing force “P” (see FIG. 3) helps counteract the pressure that results from vascular tissue engorgement. Although not wishing to be bound by any particular theory, it is believed that the invention works to alleviate pain when the external pressing force P places the sacrum and parasacral areas into a more normal anatomical relationship, thereby relaxing the pressure around the sacral foramina 126. This relaxation is believed to help restore normal venous outflow and therefore helps to relieve congestion. Accordingly, a more normal anatomy is restored, which reduces nerve irritation and decreases pain.

It has been discovered that applying pressure via a pressure distributor directly over the sacrum provides some pain relief and that applying pressure only parasacrally (to either side of the sacrum) also provides some pain relief. Furthermore, providing pressure on both the sacral and parasacral areas usually has been found to give the best relief. On the other hand, applying pressure to the lower back above the sacrum does not provide desired relief and in some cases increased pain. Accordingly, a pressure distributor according to preferred embodiments of the invention will push onto the sacral and parasacral areas but will not push as much, and preferably not at all onto the lumbar region.

As used herein the term “sacral/parasacral” means at least some of the area over the sacral bones and/or some of the area lateral to (to the left and/or right of) the line formed by the sacral bones. In preferred embodiments, the area over the sacral bones and both areas laterally (to the left and right sides of) the sacral bones and which cover at least some (preferably at least about 1 cm, 2 cm, 3 cm, 4 cm, 5 cm or more from the sacral bones) of the pelvic area are the intended targets that provide relief. Accordingly, a pressure distributor of the invention may exert pressure over the sacral region alone, the parasacral region(s) alone, but preferably would exert pressure on both the sacral and parasacral regions.

Devices according to embodiments of the invention press onto the sacral/parasacral region at least twice as hard compared to pressure applied directly over the lumbar region of the back. In some embodiments, the force is at least 5 or 10 times greater over the sacral/parasacral regions compared to the force over the lumbar region and in some embodiments is at least 20, 30, 40 or 50 times greater. In a few embodiments, the pressure distributor does not press at all upon the lumbar region. In some embodiments, a pressure distributor may be adjusted slightly above the sacral region and thus can slightly cover at least part of a lumbar vertebra as shown, for example, as outline “R” in FIG. 2. In this case as inherently described by this figure, the device still presses with substantially more force upon the sacral region than upon the lumbar region.

The Pressure Distributor

Embodiments of the invention selectively apply pressure to the sacral/parasacral region of the back by using a conforming and generally flat device herein termed a “pressure distributor.” The term “generally flat” means that the average thickness is less than one fifth the distance of any given planar dimension such as length or width and in some embodiments is less than one tenth. The term “conforming” means that, while preferably the device has some rigidity, the device adopts the surface contours of the body when used. That is, the pressure distributor is shaped or readily conforms to the epidermal contours over the sacral and/or parasacral areas of the female's body. The material will bend to conform to that same surface, particularly if more than 10 torr and, in some embodiments, if more than 20, 30, 40 50 or more torr of pressure is applied to the material. The amount of desired bend in this context will be appreciated by a user because a device that is too stiff will tend to be uncomfortable. Generally, compressible plastics and the like materials are greatly preferred for their conforming properties.

Many generally flat plastics and other like materials transmit a force exerted at a small point on one side into a much larger area in mechanical contact with the other side. Such materials are useful to make up part or all of a pressure distributor in embodiments of the invention. Based on this property, when the pressure distributor presses against the surface of a body in response to an external force at one or more points from the opposite side, the force applied initially at that one spot would be felt throughout a larger area through mechanical loading. The subsequent force applied to the body is related to the shape of the material, and for this reason, the material is termed a “pressure distributor.” The pressure distributor is advantageous for many embodiments of the invention because the pressure distributor generally will permit a tensioning apparatus, such as a tight fitting elastic garment or a belt, to apply pressure over a greater area, including typically one or more lumbar vertebrae, than the area over which the tensioning apparatus otherwise would apply pressure. The pressure distributor thus directs the force over a greater arc of the desired sacral and/or parasacral regions.

In embodiments of the invention, the pressure distributor is made of a compressible synthetic and/or non-synthetic such as a plastic other polymer and/or fiber that bends somewhat to accommodate a bend in a body contact surface. The term “bends somewhat” means that the material has a Young's modulus of elasticity between 0 and 1,000,000, although higher values might be used depending on the material (e.g. polyurethane, polystyrene, polyethylene, wood, cardboard, corrugated plastic sheet, polyvinyl chloride, foam, sponge rubber, solid rubber). Such material, when properly sized (thickness and area) and exposed to forces of, e.g., 100 torr are conforming because they will bend in response to a contour of the body. The material preferably is used at an average thickness of between 0.6 cm and 2.6 cm thick. A rubbery like material made from polyurethane or other plastic can provide acceptable results, although many other types of materials may be used. A composite such as fiberglass or a thin metal imbedded or laminated with a softer material such as one listed above also may be used.

The force distributor can be solid, or it may take the form of a gas or liquid filled chamber, honeycomb or other structure that conforms to the body surface and transmits force onto the desired (sacral/parasacral) area. The force in many embodiments arises from a tensioning apparatus, such as a tight belt, strap, set of belts or straps, tight fitting foundation garment, inflatable bladder or other apparatus that places a continuous force onto the body surface. The pressure distributor in these embodiments is interposed between the tensioning apparatus and the body, and by this position provides a more desirable and defined surface that accepts force from the tensioning apparatus. Generally, the pressure distributor must have a minimum thickness to accept force from the tensioning apparatus and distribute the force onto the sacral/parasacral region. Acceptable embodiments have, on average 0.6 cm to 2.6 cm thickness but in some cases the average thickness may exceed 2.6 cm.

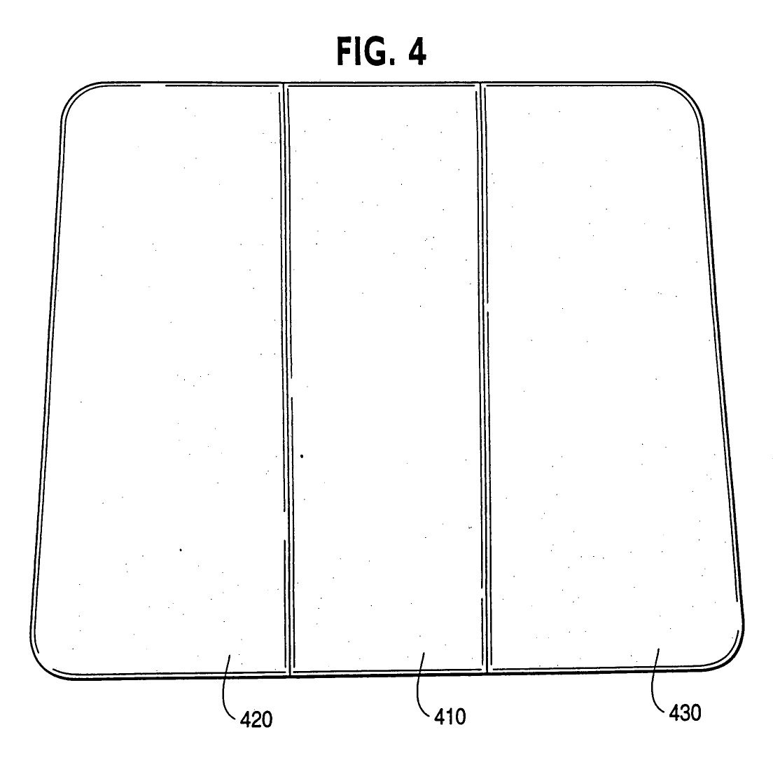

In preferred embodiments the pressure distributor is a compressible plastic that conforms to the body surface while maintaining enough thickness to distribute force from the tensioning apparatus into the sacral/parasacral region. The pressure distributor should have a minimum size to cover at least the sacral area alone or one or more parasacral areas. In some embodiments, the pressure distributor is wide enough to cover at least about 2.5 cm and more preferably at least about 5 cm or more of the parasacral area extending from each side of the sacrum as well as the sacrum area itself, without extending into the lumbar area. FIGS. 4 through 7 for example, depict an embodiment of a pressure distributor that is designed to work with a foundation garment wherein some of the elastic force of the garment is directed onto the sacral/parasacral area by stretching tightly over the pressure distributor to direct force into the desired area.

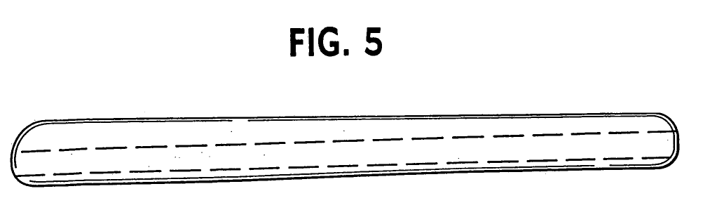

FIG. 4 is a top view of an embodiment of pressure distributor 400 wherein the top portion, which is positioned up towards the lumbar region, is narrower (14.2 cm) than the lower region (15.75 cm). The center band 410 shown in the figure is 4.4 cm wide and covers the sacrum. In certain embodiments the top is narrower than the bottom by at least 5%, 10%, 15% or more of the bottom width (FIG. 4) and the edges (shown in FIG. 5) are rounded for comfort. In certain embodiments the center 410 (resting directly over the sacrum during use) is narrower and flexible, to allow thicker regions 420 and 430 on either side to push preferentially onto the parasacral regions, after being positioned as seen by outline R in FIG. 2, with center 410 lying over the long axis of the sacral bones. The shape of such pressure distributors might be described as “moment weak” or even as a living hinge.

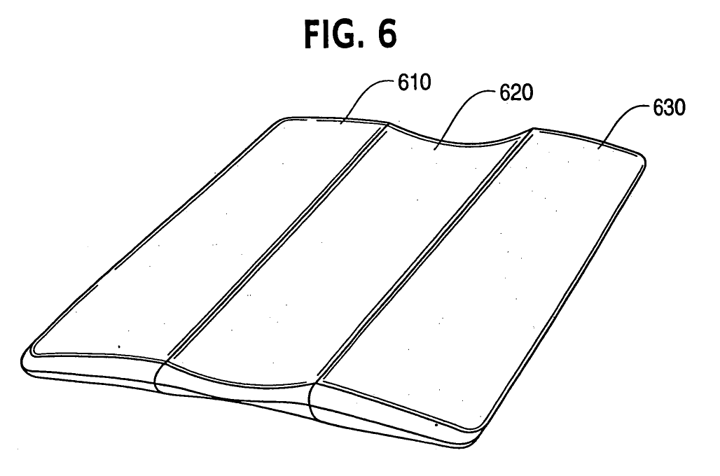

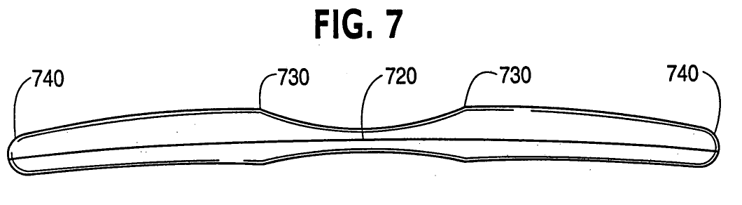

As depicted in FIG. 6 embodiments can have enough flexibility, and remain stiff such that a person can grasp the left parasacral side 610 of the device with the left hand (seen as the upper left third portion), grasp the right parasacral side 630 of the device (lower right third portion) with the right hand, and foldably bend the two sides together via flexing at the thin center 620 as seen in this figure. FIG. 7 shows an end view having a thin center that is only 0.635 cm thick at point 720 but that becomes 1.33 cm thick and stiffer at position 730 in the parasacral regions. In an embodiment the thickness reaches a maximum at position 730 but gradually decreases at further distances until a minimum is reached at position 740, which is 1.11 cm thick in the example shown. In some embodiments the pressure distributor is concave shaped, as exemplified in these figures. The concave shape facilitates application of a greater pressure on the parasacral regions than on the sacral region in one embodiment. In one such embodiment the pressure exerted on the parasacral region exceeds twice the pressure exerted on the sacral region.

The size of the pressure distributor can vary depending on the body size of the user and the type of tensioner that presses the device into the sacral/parasacral area. The device may assume a wide variety of shapes that may be placed over the sacral/parasacral region to continuously apply pressure from the tensioning apparatus. The pressure distributor may be square, polygonal or another shape. Embodiments can be four sided with bottom and top parallel to each other and sides that are between 0 and 20 degrees away from a parallel state, and in some embodiments between 2 and 10 degrees away from a parallel state. The representative shape depicted in FIG. 7 shows sides that deviate four degrees from being parallel.

Tensioning Apparatus

The pressure distributor should be able to push into the sacral/parasacral area with a force of at least 10 torr. Embodiments provide a force of at least 20, 40, 60, 80 or more and may include the ability to push with a force of at least 120, 140, 160, 180 or 200 torr or more. The force comes from a tensioning apparatus such as a belt, one or more straps, elastic foundation garment, bladder filled with air or fluid, a combination of these, or the like. The pressure distributor may be permanently coupled to the structure of the tensioning apparatus, in which case the tensioning apparatus (e.g. foundation garment, belt, etc.) is considered to include the pressure distributor as an integral component. Alternatively, the pressure distributor may be separable from the tensioning apparatus, (e.g. by a holder, such as a pocket or sleeve, velcro connector, snap connector or the like), which may be separately identifiable on the tensioning apparatus.

In some embodiments a suitable pressure distributor is selected from an assortment of alternative sizes, shapes and thickness and matched with a suitable tensioning apparatus having a holder which accepts and holds the pressure distributor in place during use. In some embodiments, the tensioning apparatus is an elastic foundation garment with a pocket for a pressure distributor, and has velcro™ straps that are adjusted to apply pressure over the pressure distributor and into the targeted sacral/parasacral areas. The user can adjust the straps to achieve optimum pain relief. Typically, the straps are adjustable to provide between about 10 and about 200 torr pressure, as measured at the skin surface between the force distributor and the skin. Embodiments, which provide from about 50 to about 175 or or from about 70 to about 120 torr pressure have been determined to be acceptable, although many other force ranges are contemplated. In such embodiments the pressure exerted onto the sacral/parasacral region is set by adjustment of one or more straps, belts, velcro fasteners, volume of air or fluid in a bladder, and the like.

The desired pushing force discussed above is exerted continuously at the sacral/parasacral area and pressure measurements are best carried out with a thin sensor interposed between the pressure distributor and the skin. A suitable pressure measuring device uses thin film resistive load cells such as the Tekscan Pressure Measurement System by Tekscan Inc. (South Boston, Mass.) with Iscan version 4.001 software (sensor part No. 9801-0937T15-T1-5-4). When measuring pressure on the sacral region for example, the sensor is positioned so that all of the sensor surface contacts, the body above the sacral bone(s).

When measuring pressure on the parasacral region, the sensor is positioned to the right side or left side of the sacral bones, such that the sensor entirely overlies the parasacral area and not over a sacral bone. A typical 7.6 cm by 12.3 cm sensor provided by Tekscan contains several smaller sensors that individually monitor pressure. If any of the smaller sensors located in the respective area indicates a given pressure value, the force distributor is determined as exerting that measured pressure force. As a practical matter, the measured values from multiple pressure sensors located within the same region (sacral or parasacral or lumbar) may be averaged to obtain a mean value for the larger set of sensors. The term “torr” as used herein means a unit of pressure, equal to 1/760 atmosphere, or 133 pascals. All values in torr are values above background atmospheric pressure and represent the added pressure from the pressure distributor.

In some embodiments a pulsating (or periodically increasing or decreasing) force in part or all of the sacral and/or parasacral regions may be provided by the pressure distributor by use of an internal mechanical mechanism such as that used typically in massaging chairs and devices. Other embodiments can provide continuous or periodic electrical stimulus to part or all of the sacral and/or parasacral regions through AC and/or DC power. Other embodiments can provide heat or cold to such areas either by electrical means or by physical addition of hot or cold liquids or objects. In this context, a “passive system” is one that exerts a steady force by virtue of a band, spring, rubber, inflated bladder or other space, elastic belt, strap and the like and does not rely on AC or DC electric power. An active system may be used that relies on AC or DC electrical power.

Foundation garments and corsets are well known and are useful for embodiments of the invention. A foundation garment or corset with a built in pressure distributor or holder for a removable pressure distributor easily may be designed in accordance with embodiments of the invention. In some cases an existing device such as a foundation garment can be modified by adding a pressure distributor. In such a case, the pressure distributor generally will exist in a thickened area underneath, i.e. on the body side of the elastic material and/or strap(s).

Tensioning may be provided by one or more bladders. In such embodiments, it may be preferred to position an elongated bladder over the left parasacral region and another elongated bladder over the right parasacral region, with the bladder axes lying parallel to the intended user's backbone. In another embodiment, the force director can be a thin (e.g., less than 0.65 cm) material with a bladder over most or all of its surface. In yet another embodiment, the bladder expands to a maximum thickness that is not constant along the surface but has a decreased thickness over the sacral area and a greater thickness in the parasacral regions in the manner shown by the center and side portions respectively of the device depicted in FIG. 7. By adding or removing fluid or gas to the bladder(s) the tension and applied force can be adjusted.

In one embodiment the pressure distributor is held at the sacral/parasacral region of a woman's lower back by a foundation garment that has a provision for holding the pressure distributor and for applying pressure to the device. For example, a panty garment may be used that has a pocket or zippered case for holding the pressure distributor. The garment may have straps, held by velcro or other fastener, which can be adjusted by the user to generate a suitable force, as determined by relief from pain. In other embodiments, the pressure distributor may be held by another article of clothing such as a belt or dress. In each case, the pressure distributor may be permanently affixed to the tensioning apparatus, may be integral with the tensioning apparatus and made from the same material, or may be releasably attached to the tensioning apparatus by, for example, velcro or by slipping the device into a pocket of the tensioning apparatus. In one embodiment the pressure distributor is a plastic that is built into the foundation garment and contains a bladder wherein adding fluid or air to the bladder causes the pressure distributor to extend and press upon the sacral/parasacral area.

The tensioning apparatus thus presses upon the pressure distributor, which in turn distributes at least some force into the sacral/parasacral area. For many embodiments, the pressure distributor will have a minimum thickness particularly over the parasacral region in order to efficiently transmit force into the parasacral region, while minimizing force to other areas. An acceptable maximum thickness generally will depend on the physical features of the intended user, but generally is between 0.6 to 3.8 cm and preferably between 1.0 to 2.2 cm. By way of example, when the pressure distributor shown in FIGS. 4 to 7 is positioned between the inside surface of a tight fitting foundation and the region of a woman's lower back over the sacral area, the elastic of the garment preferentially exerts greater force to the sacral/parasacral area than if a thinner pressure distributor were used.

In some embodiments the pressure distributor covers the sacral region and the parasacral regions only. In such case, the pressure distributor can have greatest thickness on the lateral parasacral regions and least thickness (at least one fourth less on average, at least one third less, or even at least one half less) in the center (sacral region). In other embodiments, the center (sacral region) can be thicker than the portions covering the lateral parasacral regions. Of course, in some embodiments of the invention the pressure distributor may be integrated as a portion of a garment that preferentially pushes onto the sacral/parasacral region. For example, a foundation garment may have built in indentations, a thickened region, or elongated depressions positioned over the sacral/parasacral region and that direct more tensioning force from the garment onto the sacral/parasacral region than upon the lumbar region.

The pressure distributor force preferably should substantially not distribute force to the lumbar region. The term “substantially” in this context means that any incidental pressure exerted to the nearby edge of the lumbar region (defined as the spot directly above the closest lumbar vertebra) is, on average, preferably less than 50%, more preferably less than 40%, more preferably less than 30%, more preferably less than 20%, yet more preferably less than 10% and most preferably near 0% of the average pressure force supplied to the sacral and/or parasacral areas. This preferential application of force substantially is not distributed in the lumbar region because the force director discriminates at least partly between the lumbar region and the sacral and parasacral regions. The term “at least partly” in this context means that less than half as much of the pressure distributor contacts the lumbar region compared to the surface area in contact with the sacral and parasacral regions. More preferably less than 40%, 30%, 20% or less than 10% as much of the pressure distributor contacts the lumbar region compared to the surface area in contact with the sacral and parasacral regions. Still more preferably near 0% of the surface area of the pressure distributor contacts the body directly over a lumbar vertebra.

Straps, if used, can be made from any material known to a skilled worker but those that comprise a first strip of nylon with a surface of minute hooks that fasten to a second strip containing a corresponding surface of uncut pile, such as that used in velcro™ provide good results. Other adhesive mechanisms may be used, but straps may be desirable because of their familiarity and ease of use.

All cited documents are specifically incorporated by reference in their entireties. Priority documents U.S. application Ser. No. 09/229,303, now U.S. Pat. No. 6,149,497 and 09/440,937 are incorporated by reference in their entireties.

Additional advantages and modifications will also readily occur to those skilled in the art. Therefore, the invention in its broader aspects is not limited to the specific details and representative devices, shown and described herein. Accordingly, various modifications may be made without departing from the spirit and scope of the general inventive concept as defined by the appended claims and their equivalents.

1. A device for alleviating pelvic pain in a woman, comprising a pressure distributor and a tensioning apparatus which can push the pressure distributor into the parasacral region and/or sacral region with at least 10 torr pressure while substantially not distributing force to the lumbar region of the woman's back.

2. The device of claim 1, wherein the tensioning apparatus can push the pressure distributor into the woman's back to create at least 70 torr pressure.

3. The device of claim 1, wherein the tensioning apparatus can push the pressure distributor into the woman's back to create at least 100 torr pressure.

4. The device of claim 1, wherein the tensioning apparatus is a foundation garment with a pocket for the force director, velcro pad on a foundation garment that matches velcro on a pressure distributor, a belt that permanently or reversibly affixes to the pressure distributor, a foundation garment, and/or a fabric mesh that encircles the woman's body,

5. The device of claim 1, further comprising a part for adjusting the force applied to the pressure distributor, the part consisting of an adjustable strap, a drawstring, a velcro, a solid insert, an inflatable pouch, an inflatable bladder, and/or a belt.

6. The device of claim 1, wherein the pressure distributor pushes on the parasacral region with at least five times as much force than that applied to the lumbar region.

7. The device of claim 1, wherein the pressure distributor pushes on the parasacral region with at least ten times as much force than that applied to the lumbar region.

8. The device of claim 1, wherein the pressure distributor pushes on the parasacral region with at least fifty times as much force than that applied to the lumbar region.

9. A foundation garment that preferentially applies pressure to the sacral area and/or parasacral area, while substantially avoiding the lumbar area, comprising a conforming pressure distributor positioned over the sacral and/or parasacral region wherein the pressure distributor presses upon the parasacral area when worn by a woman in need of treatment for pain.

10. The foundation garment of claim 9, wherein the pressure distributor can press upon the parasacral area with a force of at least 10 torr pressure when worn by a woman in need of treatment for pelvic pain.

11. The foundation garment of claim 9, wherein the pressure distributor can press upon the parasacral area with a force of at least 100 torr pressure when worn by a woman in need of treatment for pelvic pain.

12. The foundation garment of claim 9, wherein the pressure distributor is concave shaped and presses upon the parasacral region with a force that is at least twice the force pressing upon the sacral region.

13. The foundation garment of claim 9, wherein the pressure distributor presses upon the parasacral region with a force that exceeds by 2 fold the force pressing upon the lumbar region.

14. The foundation garment of claim 9, wherein the pressure distributor presses upon the parasacral region with a force that exceeds by 10 fold the force pressing upon the lumbar region.

15. The foundation garment of claim 9, wherein the force distributer is less than 5 centimeters thick and comprises a compressible material selected from the group consisting of plastic, foam, polyvinyl chloride, silicone, polyurethane, cellulose, synthetic fiber, laminate, fiberglass, and metal.

16. The foundation garment of claim 15, wherein the force distributer is less than 3 centimeters thick.

17. An improvement to a panty girdle, comprising:
adding a (a) pressure distributor positioned over the sacral and or parasacral region that substantially avoids the lumbar region; and a
(b) a means for adjusting pressure to the stiff portion while the panty girdle is worn.

18. A means for alleviating pelvic pain in a woman, comprising continuously pressing the sacral and/or parasacral region of the woman's back while substantially avoiding the lumbar region, through a tensioning apparatus that is designed or modified for this purpose.

19. A device for alleviating pelvic pain in a woman, comprising a conforming and generally flat pressure distributor and a tensioning apparatus, wherein the tensioning apparatus holds and continuously pushes the conforming and generally flat portion into the sacral and parasacral regions.

20. The device of claim 19, wherein the conforming and generally flat portion has a thinner area down the middle and is positioned with the thinner area straddling the sacral region of the woman's back.
